(19) United States
(12) Patent Application Publication
(10) Pub. No.: US 2023/0044007 A1
(43) Pub. Date: Feb. 9, 2023

(54) MACHINE LEARNING-SUPPORTED AND MEMORY SYSTEM-AUGMENTED SEIZURE RISK INFERENCING

(71) Applicant: Micron Technology, Inc., Boise, ID (US)

(72) Inventors: Barbara L. Casey, Star, ID (US); Carla L. Christensen, Garden Valley, ID (US); Akshaya Venkatakrishnan, Boise, ID (US); Anusha Gunda, Boise, ID (US); Yixin Yan, Boise, ID (US)

(21) Appl. No.: 17/392,385

(22) Filed: Aug. 3, 2021

(51) Int. Cl. G16H 50/30; G16H 50/20; G06N 5/04; G16H 40/63

(52) U.S. Cl. CPC G16H 50/30; G16H 50/20; G06N 5/04; G16H 40/63

(57) ABSTRACT

Methods, apparatuses, and non-transitory machine-readable media associated with seizure risk determination are described. A seizure risk determination can include receiving signaling from a radio in communication with a processing resource configured to monitor patient health data of a patient, signaling from a radio in communication with a processing resource configured to monitor health provider data associated with seizures, and signaling from a radio in communication with a processing resource configured to monitor environmental data associated with the patient. The seizure risk determination can include determining a seizure baseline for the patient and a seizure risk for the patient based on the signaling. The seizure risk determination can include identifying output data representative of a seizure plan for the patient and transmitting the output data representative of the seizure plan.

### TECHNICAL FIELD

The present disclosure relates generally to apparatuses, non-transitory machine-readable media, and methods associated with machine learning-supported and memory system-augmented seizure risk inferencing.

### BACKGROUND

Memory resources are typically provided as internal, semiconductor, integrated circuits in computers or other electronic systems. There are many different types of memory, including volatile and non-volatile memory. Volatile memory can require power to maintain its data (e.g., host data, error data, etc.). Volatile memory can include random access memory (RAM), dynamic random-access memory (DRAM), static random-access memory (SRAM), synchronous dynamic random-access memory (SDRAM), and thyristor random access memory (TRAM), among other types. Non-volatile memory can provide persistent data by retaining stored data when not powered. Non-volatile memory can include NAND flash memory, NOR flash memory, and resistance variable memory, such as phase change random access memory (PCRAM) and resistive random-access memory (RRAM), ferroelectric random-access memory (FeRAM), and magnetoresistive random access memory (MRAM), such as spin torque transfer random access memory (STT RAM), among other types.

Electronic systems often include a number of processing resources (e.g., one or more processing resources), which may retrieve instructions from a suitable location and execute the instructions and/or store results of the executed instructions to a suitable location (e.g., the memory resources). A processing resource can include a number of functional units such as arithmetic logic unit (ALU) circuitry, floating point unit (FPU) circuitry, and a combinatorial logic block, for example, which can be used to execute instructions by performing logical operations such as AND, OR, NOT, NAND, NOR, and XOR, and invert (e.g., NOT) logical operations on data (e.g., one or more operands). For example, functional unit circuitry may be used to perform arithmetic operations such as addition, subtraction, multiplication, and division on operands via a number of operations.

Artificial intelligence (AI) can be used in conjunction memory resources. AI can include a controller, computing device, or other system to perform a task that normally requires human intelligence. AI can include the use of one or more machine learning models. As described herein, the term “machine learning” refers to a process by which a computing device is able to improve its own performance through iterations by continuously incorporating new data into an existing statistical model. Machine learning can facilitate automatic learning for computing devices without human intervention or assistance and adjust actions accordingly.

### BRIEF DESCRIPTION OF THE DRAWINGS

FIG. 1 is a functional diagram representing a system for seizure risk determination in accordance with a number of embodiments of the present disclosure.

FIG. 2 is another functional diagram representing a system for seizure risk determination in accordance with a number of embodiments of the present disclosure.

FIG. 3 is a diagram representing a seizure baseline used in a seizure risk determination in accordance with a number of embodiments of the present disclosure.

FIG. 4 is another functional diagram representing a processing resource in communication with a memory resource having instructions written thereon in accordance with a number of embodiments of the present disclosure.

FIG. 5 is yet another functional diagram representing a processing resource in communication with a memory resource having instructions written thereon in accordance with a number of embodiments of the present disclosure.

FIG. 6 is a flow diagram representing an example method for seizure risk determination in accordance with a number of embodiments of the present disclosure.

### DETAILED DESCRIPTION

Systems, devices, and methods related to a seizure risk determination are described. Epilepsy is a neurological disorder marked by sudden recurrent episodes of sensory disturbance, loss of consciousness, or convulsions called seizures. The seizures can be associated with abnormal electrical activity in the brain. Some wearable devices can detect patterns that may be associated with a convulsive seizure called a tonic-clonic, but other seizure types including focal onset seizures and generalized onset seizures may not be detected.

Examples of the present disclosure can allow for customized, individual early stress detection to determine a seizure risk and seizure plan to potentially prevent seizures and understand seizure triggers. Examples can include the use of a machine learning model or models utilizing real-time and ad-hoc patient health data, information from the medical field, and environmental data to determine a seizure risk and a seizure plan.

Examples of the present disclosure can include a method for determining a seizure risk including receiving at a first processing resource, first signaling from a radio in communication with a second processing resource configured to monitor patient health data of a patient and receiving at the first processing resource, second signaling from a radio in communication with a third processing resource configured to monitor health provider data associated with seizures. Examples can include receiving at the first processing resource, third signaling from a radio in communication with a fourth processing resource configured to monitor environmental data associated with the patient and writing from the first processing resource to a memory resource coupled to the first processing resource data that is based at least in part on a combination of the first signaling, the second signaling, and the third signaling.

Examples can include determining, at the first processing resource or a different, fifth processing resource, a seizure baseline for the patient and a seizure risk for the patient based on the first signaling, the second signaling, and the third signaling and identifying, at the first processing resource or the different, fifth processing resource, output data representative of a seizure plan for the patient based at least in part on input data representative of written information, the seizure baseline, and the seizure risk stored in a portion of the memory resource or other storage accessible by the first processing resource. Examples can include transmitting the output data representative of the seizure plan via fourth signaling sent via a radio in communication with a sixth processing resource of a computing device accessible by the patient.

In the following detailed description of the present disclosure, reference is made to the accompanying drawings that form a part hereof, and in which is shown by way of illustration how one or more embodiments of the disclosure can be practiced. These embodiments are described in sufficient detail to enable those of ordinary skill in the art to practice the embodiments of this disclosure, and it is to be understood that other embodiments can be utilized and that process, electrical, and structural changes can be made without departing from the scope of the present disclosure.

It is also to be understood that the terminology used herein is for the purpose of describing particular embodiments only and is not intended to be limiting. As used herein, the singular forms “a,” “an,” and “the” can include both singular and plural referents, unless the context clearly dictates otherwise. In addition, “a number of,” “at least one,” and “one or more” (e.g., a number of memory devices) can refer to one or more memory devices, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to.” The terms “coupled,” and “coupling” mean to be directly or indirectly connected physically or for access to and movement (transmission) of commands and/or data, as appropriate to the context.

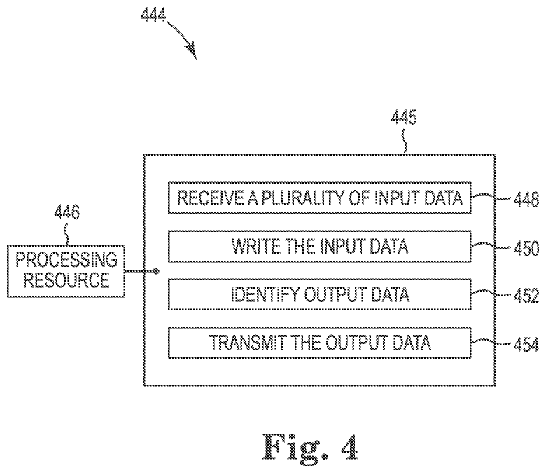

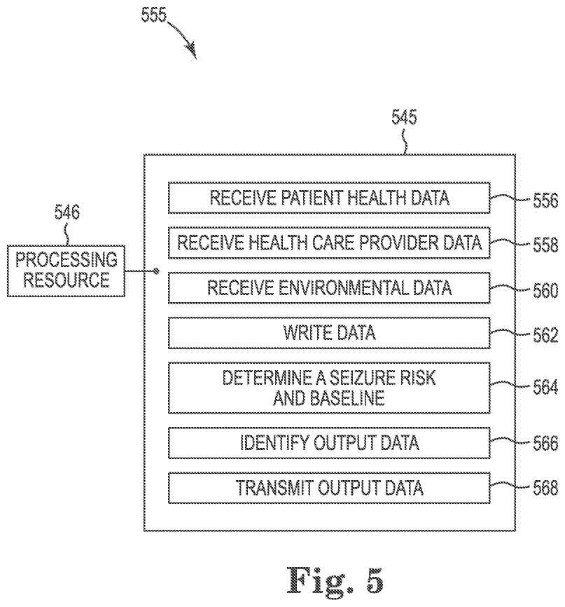

The figures herein follow a numbering convention in which the first digit or digits correspond to the figure number and the remaining digits identify an element or component in the figure. Similar elements or components between different figures can be identified by the use of similar digits. For example, 446 can reference element “46” in FIG. 4, and a similar element can be referenced as 546 in FIG. 5. As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

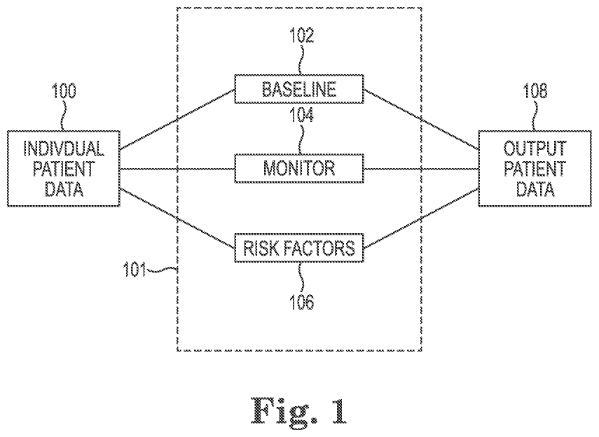

FIG. 1 is a functional diagram representing a system for seizure risk determination in accordance with a number of embodiments of the present disclosure. The system can include a seizure risk tool 101 used to determine a seizure risk, seizure plan, or both, for a patient who has experienced a seizure. The seizure risk can include a likelihood at a particular period or point in time that the patient will have a seizure, and the seizure plan can include a plan of action, both immediate and long-term to reduce the seizure risk.

The seizure risk tool 101 can include, in some examples, a processing resource in communication with a memory resource that utilizes AI to determine a seizure risk, a seizure plan, or both. Put another way, the seizure risk tool 101 and associated AI determines a seizure risk and/or creates a plan of action for a patient based on data available to the seizure risk tool 101 including, but not limited to, patient health data, health care provider data, and environmental data.

The seizure risk tool 101 (and associated AI (e.g., including machine learning model(s)) can be trained using a training dataset. For instance, the training dataset can include a set of examples used to fit parameters of the AI. For instance, the training dataset can include data associated with patient health data, health care provider data, and environmental data, among others. In some examples, the seizure risk tool 101 can also be trained using new input data (e.g., new data from patients, providers, environmental sensors, research data, etc., among others). In some examples, the seizure risk tool 101 and associated trained machine learning model can include continuous learning of the machine learning model and re-calibration of the machine learning model.

The seizure risk tool 101 can receive input data from a plurality of sources. The input data can be encrypted, in some examples. Sources can include a database of generic seizure patient and/or treatment information, patient health information sources (e.g., personal tracking devices, personal medical devices, insurance information, patient health data, etc.), providers (e.g., health care provider data), and environmental information sources (weather sensors, cameras, motion detectors, light detectors, diet sensors, etc.). For instance, the database of generic seizure patient and/or treatment information may include common symptoms, visuals, treatments, and other data associated with seizures.

Patient data can be received from personal tracking devices such as a global positioning service (GPS) on a mobile device including a patient location, a sleep tracking application, dietary tracking application, fitness tracking application, or other personal tracking device, among others. Patient health data can be received from personal medical devices in real time (e.g., heartrate monitor, sleep monitors, oxygen level monitors, etc.). In some examples, patient health data can be received ad-hoc, for instance as data from a health care provider (e.g., blood test results, vital signs, physician notes, age, medical history, etc.) or a patient can manually input patient data such as address or birthday information and/or patient health data such as current symptoms, current ailments, medication tracking, family health history, allergies, patient health history, etc. via an application on a computing device and associated with the seizure risk tool 101.

For instance, at 100, individual patient data can be monitored and collected. For instance, patient health data can be monitored, using a health sensor, health monitor, wearable device, mobile device, etc. of the patient. A patient's heartrate, blood pressure, skin conductivity, cortisol levels, skin gasses, body temperature, oxygen levels, sleep patterns, motions (e.g., shaking), diet, exercise patterns, exercise levels, and other data may be monitored. In some examples, health care provider data can be received including, for instance vital signs from previous visits, test results (e.g., blood mapping), past seizure types, age, and medical history, among others. In some examples, environmental data (e.g., humidity, lighting, temperature, etc.) can be received at the seizure risk tool 101 as part of the individual patient data 100.

At 102, a seizure baseline can be established using the seizure risk tool 101. A seizure baseline, as used herein, can include a dynamic (e.g., changeable as new data is received) seizure baseline that indicates when a patient may experience a seizure. For instance, the seizure risk tool 101 can receive input data representative of patient health data, health care provider data, and environmental data from a plurality of sources. Based on the received input data, the seizure risk tool 101 can determine conditions under which the patient is at a highest risk for seizure.

The seizure baseline can be established at a first seizure of the patient and updated as more input data is received and in response to subsequent seizures. For instance, a patient experiencing high stress levels and high fever may be above a seizure baseline and at a higher risk of seizure as compared to the same patient when he or she is calm and fever-free. Example triggers that may be monitored or submitted by a patient (e.g., via an application) include particular times of the day or night, sleep deprivation (overtired, not sleeping well, insufficient sleep, etc.), fever, illness, flashing lights, patterns, alcohol use, drug use, stress, menstrual cycle, hormonal changes, poor eating habits, low or high blood sugar, particular foods, caffeine, and/or medications, among others.

At 104, deviations from the seizure baseline can be monitored and flagged. In the previous example, the patient with a fever deviates from a seizure baseline with a high temperature. For instance, a temperature sensor on a wearable device may transmit temperature levels to the seizure risk tool 101 in real time. The seizure risk tool 101 can monitor these levels and determine when the patient is deviating from the seizure baseline.

At 106, risk factors associated with the patient can be identified. For example, the seizure risk tool 101 can receive and consider environmental data and health care provider data to determine potential seizure risk factors for the patient. For instance, in a non-limiting example, health care provider data may indicate the patient has similar health conditions to other patients in medical research data who experience seizure in high humidity environments. The seizure risk tool 101 can receive input data representing humidity levels (e.g., via weather sensors, wearable devices, etc.) and monitor the input data and potential seizure risk. Other seizure risk factors may be monitored and considered.

In some examples, using generic seizure patient information, generic seizure treatment information, patient medical history information, or any combination thereof, a profile for the patient and/or other generic patient profiles can be created. The patient's seizure baseline can be mapped to the patient profile for adjustment as changes occur. Using the seizure baseline, deviations, risk factors, patient profile, generic seizure patient information, generic seizure treatment information, patient medical history information, or any combination thereof, output patient data 108 representative of a seizure risk and a seizure plan can be determined, transmitted, or both, using the seizure risk tool 101. Output patient data 108, in some instances, can include warning transmissions, to a computing device of a patient, physician, or authorized user to alert the patient of a seizure risk and provide a seizure plan. In some examples, output patient data 108 can include an alert sent to a non-mobile device such as a television screen, personal computer, refrigerator display, or smart device (e.g., smart speaker), among others.

In some examples, as will be discussed further herein, different sources and associated data may be assigned different weights within the seizure risk tool 101. For instance, a source determined to provide data more likely to predict a seizure may be given more weight than a source determined to provide data less likely to predict a seizure. The seizure risk and the seizure plan, as will be discussed further herein, can be used to assist a patient to reduce a risk of experiencing a seizure, alert a user if a seizure potential is high, or both.

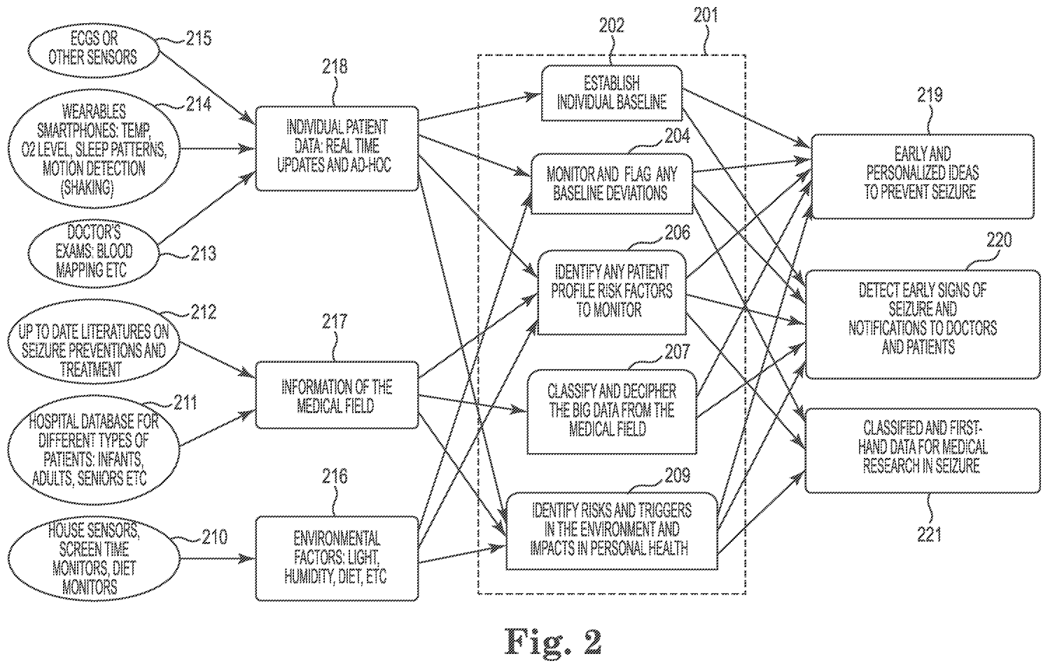

FIG. 2 is another functional diagram representing a system for seizure risk determination in accordance with a number of embodiments of the present disclosure. The system can include a seizure risk tool 201 that may be analogous to seizure risk tool 101, device 444, and/or device 555 illustrated in FIGS. 1, 4, and 5, respectively.

The seizure risk tool 201 can receive individual patient data at 218, which can be received at the seizure risk tool 201 as real time or ad-hoc updates. The individual patient data, referred to herein as “patient health data,” can include health data specific to the patient, and can be received from a plurality of sources including, for instance, sensors at 215, wearable devices or other smart devices (e.g., smartphones) at 214, and medical examinations at 213. The sensors can include devices that detect or measure a physical property and record, indicate, or otherwise report it. An example sensor is an electrocardiogram (ECG). The wearable devices or other smart device can include sensors, in some examples, and can use those sensors to gather data including body temperatures, oxygen levels, sleep patterns, changes in motion or other motion (e.g., shaking), among others. The medical examination information can include data collected at a healthcare provider's (e.g., doctor) office or exam such as blood mapping, vital signs, weight etc.

At 217, the seizure risk tool 201 can receive information associated with the medical field, referred to herein as “health care patient data”. This can include, for instance, receiving up-to-date literatures on seizure preventions and treatments at 212 or gathering data from a hospital or other database for different types of patients at 211, such as infants, adults, seniors, patients with particular conditions, etc. At 216, the seizure risk tool 201 can receive data associated with environmental factors, referred to herein as “environmental data”, including for instance light, humidity, temperatures, eating habits, etc. This data can be gathered using sensors at 210 including, for instance, sensors around the home (e.g., cameras, motion detectors, etc.), temperature sensors, screen time monitors, diet monitors, etc.

As indicated by the arrows, different input data 218, 217, 216 can be used by the seizure risk tool 201 to made different determinations. For instance, an individual seizure baseline can be established at 202 using patient health data 218. The seizure risk tool 201 can utilize a machine learning model that considers the patient health data 218 to establish a seizure baseline 202 including an estimated threshold for the patient at which a seizure becomes more likely. For instance, the machine learning model, which can be a trained machine learning model, can consider when a patient's first seizure occurred, as well as any subsequent seizures, and determine what occurred when those seizures occurred. The machine learning model can use this information, along with any historic patient health data, newly received ad-hoc patient health data, and real time received patient health data to establish and update the seizure baseline.

At 204, deviations from the seizure baseline 202 can be monitored and flagged using patient health data 218 and environmental data 216. For instance, as real time data and ad hoc data is received, whether patient health data or environmental data, the machine learning model can monitor it and determine if it deviates from the seizure baseline. For instance, if input data associated with a patient's blood pressure is received at the seizure risk tool 201, and the machine learning model indicates it deviates from the seizure baseline (e.g., too high), the blood pressure and time can be flagged. Similar, if input data representative of screen time received at the seizure risk tool is determined by the machine learning model to be too high, the situation can be flagged. In some examples, one or more deviations may result in a change to the seizure baseline, a transmitted seizure risk warning, or both.

At 206, risk factors associated with the patient can be identified using patient health data 218, health care provider data 217, and environmental data 216. For instance, input data representative of patient health data 218 may indicate a patient has consistently rising blood pressure. The machine learning model may detect this as a risk factor of the patient and adjust a weight factor of blood pressure when determining a seizure baseline, request more frequent monitoring, or both. Similar, the machine learning model may consider information from a health journal (e.g., health care provider data 217) indicating that a particular amount of screen time may affect seizure frequency. The machine learning model may detect this as a risk factor of the patient, consider historic screen time data (e.g., received as environmental data 216), adjust a weight factor of screen time when determining a seizure baseline, and request more frequent monitoring, or any combination thereof.

At 207, the health care provider data 217, including big data from the medical field can be classified and deciphered. For instance, the machine learning model may consider data received from health journals, press articles, medical research, etc., and determine how the data applies to the patient. For instance, the machine learning model may disregard or assign a low weight to input representative of medical research performed only on young men when the patient is an elderly woman.

At 209, risks and triggers in the environment and how they may impact the patient's personal health can be identified using patient health data 218, health care provider data 217, and environmental data 216. For instance, environmental risks and/or triggers may include quick temperature changes, screen time, certain foods, medications, light intensity, and humidity, among others. Based on historic and current patient data, historic and current environmental data, and historic and current health care provider data, the machine learning model can determine when environmental factors have affected the patient, to what extent the environment factors affected the patient, whether the environmental factors occurred within a threshold time of a seizure, and how different areas of the patient's health were affected by the environmental factors.

The seizure risk tool 201 can output data representative of a seizure risk, seizure plan, or both, based on the various determinations made by the seizure risk tool 101 using machine learning. For instance, at 219, early and personalized ideas to prevent seizure can be provided to a computing device of the patient, health care provider, or authorized user. The ideas 219 can be determined using the established individual seizure baseline 202, baseline deviations 204, patient risk factors 206, classified and deciphered healthcare provider data 207, and environmental risks and triggers 209.

For example, the machine learning model may determine a blood pressure deviation from the seizure baseline 202. Based on other determinations made by the machine learning model, the output 219 may include a warning to a computing device of the patient, physician, or other authorized user that a seizure risk is high because of the deviation. A recommendation to the patient to lie down in a dark room and practice breathing exercises may be made, for instance, based on determined risk factors, environmental triggers, deciphered big data, etc. Other recommendations and warnings may be transmitted.

At 220, the seizure risk tool 201 can detect early signs of seizure, for instance using machine learning, and provide notifications to a computing device of the patient, health care provider, or authorized user. This detection, for instance, can be determined using the established individual seizure baseline 202, baseline deviations 204, patient risk factors 206, classified and deciphered healthcare provider data 207, and environmental risks and triggers 209.

For instance, a wearable device or other sensor of the patient may detect a particular environmental humidity, patient stress level, and patient blood sugar. The machine learning model may determine the combination of these levels may raise the patient above the seizure baseline 202. Similar, the machine learning model may determine a combination of rising blood pressure and stress levels are leading the patient towards a potential seizure. Such determinations and detections can be transmitted to a computing device of the patient, a physician, or other authorized user.

At 221, the seizure risk tool 201 can prepare, for instance using machine learning, classified and first-hand data for medical research in the seizure field. Using baseline deviations 204, patient risk factors 206, and environmental risks and triggers 209, data can be collected, classified, and transmitted to a computing device for use in medical research. For instance, based on the data collected and analyzed by the seizure risk tool 201 and associated AI, data can be transmitted to medical professionals or researchers, among others, for use in medical research.

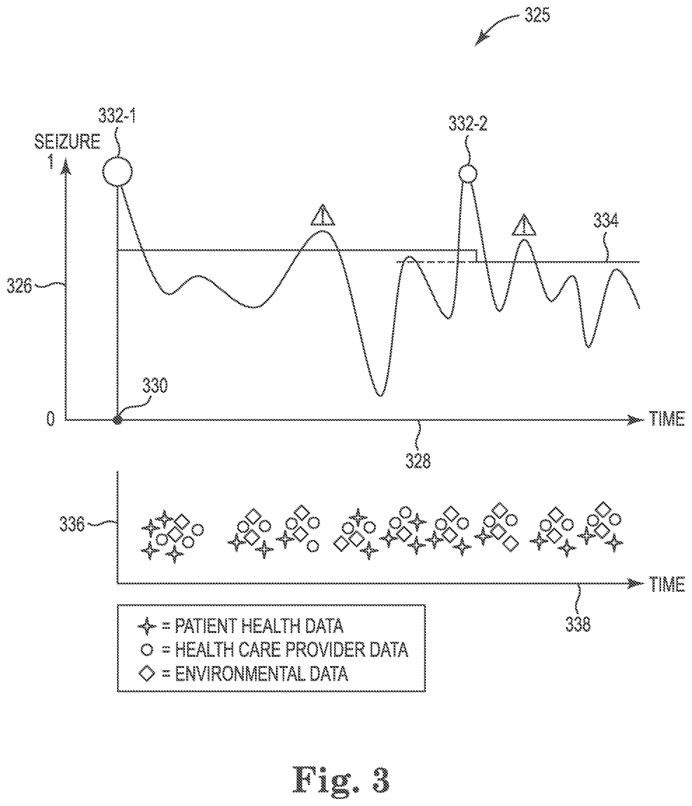

FIG. 3 is a diagram 325 representing a seizure baseline 334 used in a seizure risk determination in accordance with a number of embodiments of the present disclosure. The diagram 325 includes a likelihood of a seizure 326 over time 328, with the seizure baseline 334 acting as a reference line. A zero on the seizure likelihood axis 326 indicates little to no chance the patient will experience a seizure. A one on the seizure likelihood axis 326 indicates a seizure occurred.

The seizure baseline 334 can be established using a seizure risk tool that considers patient health data, health care provider data, and environmental data. For instance, the patient health data, health care provider data, and environmental data are inputs 336 received at the seizure risk tool over time 338. In some examples, the time 338 is the same as the time 328. The patient health data and the environmental data can be received in real time or ad hoc, and the health care provider data can be received periodically (e.g., as new data becomes available). Each input 336 can be weighted, with some inputs 336 (e.g., particular patient health data) carrying a greater weight than other inputs 336 (e.g. particular health care provider data). In some examples, the inputs 336 carry the same weights.

The inputs can begin, for instance, at the first seizure 332-1 experienced by a patient at a particular age 330. As time 328, 338 progresses, inputs 336 can be received at a seizure risk tool, and based on those inputs 336, the seizure risk tool and associated AI (e.g., including machine learning model(s)) can determine a seizure risk. For instance, if a machine learning model indicates the patient's seizure risk is above the seizure baseline (e.g., at the triangle warning symbols), a warning may be transmitted to a computing device of the patient or other authorized user to implement a seizure plan, whereas if the machine learning model indicates the patient's seizure risk is below the seizure baseline, a warning may not be transmitted, but a periodic report transmitted to a computing device of the patient or other authorized user may include the seizure risk over time 328, 338.

In some examples, a patient may experience a second seizure, as indicated at 332-2. In such an example, the seizure plan tool can receive input data 336, and the machine learning model can be adjusted accordingly, along with the seizure baseline 334, as indicated by the dashed line. For example, the seizure baseline 334 can be lowered responsive to the second seizure 332-2. Each time a patient experiences a seizure, the seizure baseline 334 and associated machine learning models can be adjusted. The seizure baseline 334 can also be adjusted in response to additional inputs 336. For instance, as more input data 336 is received at the seizure risk tool from the patient, a physician, medical research, etc., the seizure baseline can adjust accordingly.

When the patient's seizure risk nears the seizure baseline 334 or rises above it, the patient can be notified to take action to reduce a risk of seizure. For instance, a warning may be transmitted to a computing device (e.g., smartphone, smartwatch, etc.) of the patient, physician, other authorized user, or any combination thereof suggesting the patient take action to reduce stress (e.g., turn off lights, sit down, take deep breaths, avoid other potential triggers, etc.).

FIG. 4 is another functional diagram representing a processing resource 446 in communication with a memory resource 445 having instructions 448, 450, 452, 454 written thereon in accordance with a number of embodiments of the present disclosure. In some examples, the processing resource 446 and the memory resource 445 comprise a device 444 and may be analogous to device 555 illustrated in FIG. 5, seizure risk tool 101 illustrated in FIG. 1, and/or seizure risk tool 201 illustrated in FIG. 2.

The device 444 illustrated in FIG. 4 can be a server or a computing device (among others) and can include the processing resource 446. The device 444 can further include the memory resource 445 (e.g., a non-transitory MRM), on which may be stored instructions, such as instructions 448, 450, 452, 454. Although the following descriptions refer to a processing resource and a memory resource, the descriptions may also apply to a system with multiple processing resources and multiple memory resources. In such examples, the instructions may be distributed (e.g., stored) across multiple memory resources and the instructions may be distributed (e.g., executed by) across multiple processing resources.

The memory resource 445 may be electronic, magnetic, optical, or other physical storage device that stores executable instructions. Thus, the memory resource 445 may be, for example, non-volatile or volatile memory. In some examples, the memory resource 445 is a non-transitory MRM comprising RAM, an Electrically-Erasable Programmable ROM (EEPROM), a storage drive, an optical disc, and the like. The memory resource 445 may be disposed within a controller and/or computing device. In this example, the executable instructions 448, 450, 452, 454 can be “installed” on the device. Additionally, and/or alternatively, the memory resource 445 can be a portable, external or remote storage medium, for example, that allows the system to download the instructions 448, 450, 452, 454 from the portable/external/remote storage medium. In this situation, the executable instructions may be part of an “installation package”. As described herein, the memory resource 445 can be encoded with executable instructions for seizure risk determination.

The instructions 448, when executed by a processing resource such as the processing resource 446 can include instructions to receive at the processing resource 446, the memory resource 445, or both, a plurality of input data from a plurality of sources, the plurality of sources comprising at least two of: a mobile device of a patient, a medical device, a health care provider database, a portion of the memory resource or other storage, manually received input, and environmental sensors. In some examples, the plurality of sources can include computing device data, application data (e.g., diet monitoring application, fitness application, etc.), which may be stored on the mobile device, the memory resource, the other storage, or a combination thereof. The plurality of input data, for instance, can include patient health data, health care provider data, environmental data, or any combination thereof.

For example, the processing resource 446, the memory resource 445, or both can receive health data specific to the patient (e.g., heartrate, blood pressure, vital signs, weight, etc.) in an ad-hoc or real time manner as patient health data. This patient health data may come from a wearable device or other device of the patient, from a health care provider, or as manual input (e.g., via an application). The processing resource 446, the memory resource 445, or both can receive as health care provider data medical research data, publication data, big data, etc. associated with seizures. This data, for instance, may come from generic databases of seizure data (e.g., common triggers, factors among age groups, etc.), among other sources. In some examples, the processing resource 446, the memory resource 445, or both can receive environmental data from sensors or other sources including, for instance, dietary information, screen time information, humidity information, light information, etc.

The instructions 450, when executed by a processing resource such as the processing resource 446 can include instructions to write from the processing resource 446 to the memory resource 445 the received plurality of input data, and the instructions 452, when executed by a processing resource such as the processing resource 446 can include instructions to identify at the first processing resource 446 or a second processing resource, output data representative of a seizure plan including a proposed action to reduce a seizure risk of the patient, a proposed action to stay at or below a seizure baseline of the patient, or both, based at least in part on input data representative of the data written from the first processing resource 446. For instance, the seizure plan can be dependent on a seizure risk of a patient using a seizure baseline established by the machine learning model.

In some examples, identifying the output data representative of the seizure plan can be based at least in part on generic seizure patient information, generic seizure treatment information, patient medical history information, or any combination thereof stored in a portion of the memory resource 445 or other storage (e.g., additional memory resource, cloud storage, etc.) accessible by the first processing resource 446. Put another way, a machine learning model (e.g., a trained machine learning model) can determine the output data, and the memory resource 445 or other storage can include databases of information accessible by the processing resource 446 for use in the machine learning model. In some examples, the database information may be used to train the machine learning model.

The instructions 454, when executed by a processing resource such as the processing resource 446 can include instructions to transmit the output data representative of the seizure plan to the mobile device of the patient via signaling sent via a radio in communication with a third processing resource of the patient's mobile device. For instance, a user can receive a warning at a smartphone, smartwatch, or other device to take action to reduce stress to avoid a seizure. As used herein, the use of a radio can include the transmission and/or reception of information through intervening media (e.g., air, space, nonconducting materials, etc.). This can include, for instance, radio waves or other wireless communication and/or signaling including but not limited to cellular communication, one-way communication, two-way communication, radar, radiolocation, radio remote control, satellite communication, Wi-Fi, 3G, 4G, 5G, and/or other communication standards, among others. In some examples, the use of a radio can include wired transmission and/or reception of information.

In some examples, the instructions 454, when executed by a processing resource such as the processing resource 446 can include instructions to transmit an alert to the mobile device of the patient of the seizure risk, the proposed action to reduce the seizure risk of the patient, the proposed action to stay at or below the seizure baseline of the patient, or any combination thereof. For example, if the machine learning model determines the patient is at risk for a seizure, this risk, along with the seizure plan (e.g., turn off the lights, sit down, breath deeply, etc.) can be transmitted to the mobile device of the patient, a physician, an authorized user (e.g., spouse, parent, etc.), or any combination thereof.

FIG. 5 is yet another functional diagram representing a processing resource 546 in communication with a memory resource 545 having instructions 556, 558, 560, 562, 564, 566, 568 written thereon in accordance with a number of embodiments of the present disclosure. In some examples, the processing resource 546 (herein after referred to as the first processing resource 546) and the memory resource 545 comprise a device 555 and may be analogous to device 444 illustrated in FIG. 4, tool 101 illustrated in FIG. 1, and/or tool 201 illustrated in FIG. 2.

The instructions 556, when executed by a processing resource such as the first processing resource 546 can include instructions to receive at the first processing resource 546, the memory resource 545, or both, patient health data via first signaling configured to monitor patient health data, via second signaling sent via a radio in communication with a processing resource of a mobile device of the patient, or both. The first signaling may be received from a health sensor, health monitor, wearable device, mobile device of the patient, or any combination thereof. For instance, this first signaling can include real time patient health data such as a heartrate, blood pressure, or blood sugar level, among others. The second signaling may be received from the mobile device of the patient. For instance, the second signaling can include manually input data (e.g., via an application) such as age, weight, height, physician information, etc. In some instance, patient health data may be received from a health care provider (e.g., vitals, bloodwork results, etc.). The patient health data can include health symptoms, a health event (e.g., seizure, surgery, heart attack, etc.), personal health information of the patient, identifying information of the patient, a location of the patient, data collected by a health monitor, manually input data of the patient, or any combination thereof.

The instructions 558, when executed by a processing resource such as the first processing resource 546 can include instructions to receive at the first processing resource 546, the memory resource 545, or both, health care provider data via third signaling configured to monitor health care provider data including generic seizure patient information and generic seizure treatment information. For instance, the health care provider data can include data associated with medical research or treatment databases including common and rare seizure triggers, seizure treatments, or seizure trends in genders, ages, among others.

The instructions 560, when executed by a processing resource such as the first processing resource 546 can include instructions to receive at the first processing resource, the memory resource, or both, environmental data via fourth signaling configured to monitor environmental data including lighting, screen time, diet, humidity, temperature, or any combination thereof. The environmental data, for instance, can be collected using environmental sensors such as temperature or other weather sensors, screen time sensors, food tracking sensors, lighting sensors, etc.

The instructions 562, when executed by a processing resource such as the first processing resource 546 can include instructions to write from the first processing resource to the memory resource the patient health data, heath care provider data, and environmental data. In some examples, the memory resource 545 or other storage can include a database including generic seizure symptoms and associated diagnoses and treatments. The other storage, in some examples, may include cloud storage (e.g., secure cloud storage).

The instructions 564, when executed by a processing resource such as the first processing resource 546 can include instructions to determine, at the first processing resource 546 or a second processing resource, a seizure risk of the patient and a seizure baseline for the patient using a trained machine learning model, input data representative of the written patient health data, the written heath care provider data, and the written environmental data. Put another way, using the machine learning model, a probability the patient will experience a seizure and at what point that may occur is determined. The seizure baseline can consider several factors associated with the patient to determine a set of circumstances most likely to precede a seizure. Deviations from the seizure baseline may indicate a seizure and are flagged by the machine learning model.

In some examples, the patient health data, the health care provider data, and the environmental data carry different weights within the trained machine learning model. For instance, patient health data may be given a great weight than health care provider data, as the patient health data is specific to the patient. The weights can change as more data is received and the machine learning model is updated. For example, if the patient experiences a seizure immediately after being exposed to flashing lights, all or some environmental factors may be given a higher weight.

The instructions 566, when executed by a processing resource such as the first processing resource 546 can include instructions to identify, at the first processing resource 546 or a second processing resource, output data representative of a seizure plan for the patient using the trained machine learning model, input data representative of the written patient health data, the written health care provider data, and the written environmental data, and input data representative of the seizure risk and the seizure baseline. The seizure plan can include a seizure risk and a plan of action for addressing the seizure risk. For instance, the seizure plan can include how to immediately address the risk, as well as an ongoing plan to address the seizure risk and/or potential seizure triggers.

In some examples, the instructions 566 can be executable to determine an alert to transmit to the mobile device of the patient of the seizure risk, determine an alert to transmit to a computing device of a health care provider of the seizure risk, determine an alert to transmit to a mobile device of an authorized user of the seizure risk, determine a proposed action to reduce the seizure risk of the patient, determine a proposed action to stay at or below the seizure baseline of the patient, or any combination thereof. Put another way, the seizure plan can include warnings of a seizure risk, suggestions of who should know of the seizure risk, and proposed actions to address the seizure risk.

The instructions 568, when executed by a processing resource such as the first processing resource 546 can include instructions to transmit, via a radio, the output data representative of the seizure plan to the patient, a health care provider, or any combination thereof. For instance, a patient may receive an immediate alert if a determination is made that the patient is at a high seizure risk or may receive periodic updates if it is determined the patient is at a low seizure risk. For instance, if the patient is experiencing a threshold number of potential triggers indicating a rise above the seizure baseline, the patient may receive an audio, physical, or other alert at a mobile device including a seizure risk and a seizure plan to reduce stress to avoid a potential seizure.

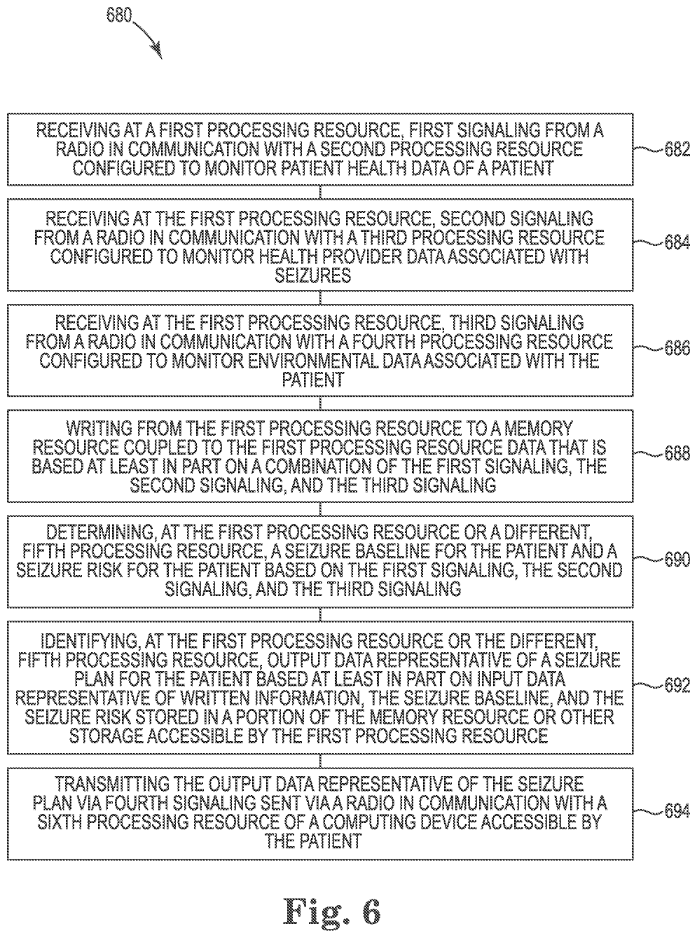

FIG. 6 is a flow diagram representing an example method 680 for seizure risk determination in accordance with a number of embodiments of the present disclosure. The method 680 may be performed, in some examples, using a seizure risk tool 101, 201 and/or a device such as devices 444, 555 as described with respect to FIGS. 1, 2, 4, and 5.

The method 680, at 682, can include receiving at a first processing resource, first signaling from a radio in communication with a second processing resource configured to monitor patient health data of a patient. For instance, the second processing resource may include a sensor for monitoring patient health data such as oxygen levels, heartrates, body temperature, etc. Patient health data may include, in some instances, data from a health care provider visit (e.g., bloodwork, vital signs, etc.).

At 684, the method 680 can include receiving at the first processing resource, second signaling from a radio in communication with a third processing resource configured to monitor health provider data associated with seizures. Health provider data, for instance, can include medical research and/or databases of generic seizure symptom, seizure trigger, and/or seizure treatment data. For instance, this can include “big data” compiled by a health care provider or other source for different patients.

At 686, the method 680 can include receiving at the first processing resource, third signaling from a radio in communication with a fourth processing resource configured to monitor environmental data associated with the patient. For instance, the fourth processing resource may include a sensor for monitoring environmental data such as temperature data, lighting data, humidity levels, etc.

The method 680, at 688, can include writing from the first processing resource to a memory resource coupled to the first processing resource data that is based at least in part on a combination of the first signaling, the second signaling, and the third signaling. The written data can be saved at the memory resource for use in determination of a current or future seizure plan.

At 690, the method 680 can include determining, at the first processing resource or a different, fifth processing resource, a seizure baseline for the patient and a seizure risk for the patient based on the first signaling, the second signaling, and the third signaling. The seizure baseline can include levels of each one of a plurality of factors associated with the patient at which a seizure is most likely to occur. For instance, the seizure baseline may include a blood pressure at level A, a body temperature at level B, and a screen time level of C. Deviations from the seizure baseline may be monitored and flagged. The seizure risk can include a likelihood at a particular period in time that the patient will have a seizure. For instance, if the patient deviates above the seizure baseline, the seizure risk may be high for the patient.

In some examples, determining the seizure baseline and the seizure risk can include utilizing a trained machine learning model to determine the seizure baseline and the seizure risk based on data associated with the first signaling, the second signaling, the third signaling, and previously received signaling and associated data associated with previous seizure plans. For instance, as data is received at the machine learning model, the baseline is updated, along with the seizure risk. If a previous seizure plan had elements that worked and elements that did not, the seizure plan, seizure baseline, and seizure risk can be updated as new and updated data are received at the machine learning model. For instance, the seizure baseline can be updated in response to receiving at the first processing resource additional first signaling, second signaling, third signaling, or any combination thereof and based at least in part on feedback received at the first processing resource associated with outcomes of the output data representative of the seizure plan.

At 692, the method 680 can include identifying, at the first processing resource or the different, fifth processing resource, output data representative of a seizure plan for the patient based at least in part on input data representative of written information, the seizure baseline, and the seizure risk stored in a portion of the memory resource or other storage accessible by the first processing resource. In some examples, identifying the output data representative of the seizure plan includes utilizing a trained machine learning model to identify the output data representative of the seizure plan based on data associated with the first signaling, the second signaling, the third signaling, the seizure baseline, the seizure risk, and previously received signaling and associated data associated with previous seizure plans. The seizure plan, for instance, can include the seizure risk and an associated plan to address the risk including, for instance, actions to take to reduce stress (e.g., turn of lights, take a nap, drink water, etc.). The seizure plan can be transmitted to the patient, physician, authorized user, or any combination thereof.

For instance, identifying the output data representative of the seizure plan can include identifying an alert to transmit to the computing device of the patient and identifying a proposed action and associated instructions to reduce a seizure risk of the patient, a proposed action and associated instructions to stay at or below a seizure baseline of the patient, or both. The method 680, at 694, can include transmitting the output data representative of the seizure plan via fourth signaling sent via a radio in communication with a sixth processing resource of a computing device accessible by the patient. For instance, the identified alert can be transmitted to the computing device accessible by the patient (e.g., smartphone, wearable device, etc.). In some instances, the identified alert can be transmitted to a computing device or other mobile or non-mobile device of an authorized user (e.g., caregiver, guardian, etc.) or health care provider.

In some examples, the method 680 can include receiving at the first processing resource via an application of the computing device accessible by the patient or a different a mobile device of the patient, manual input from the patient comprising personal patient data, patient health data, environmental data, health care provider data, or a combination thereof and writing from the first processing resource to the memory resource coupled to the first processing resource data that is based at least in part on a combination of the first signaling, the second signaling, the third signaling, and the manual input. Put another way, the patient's seizure risk, seizure baseline, and seizure plan can be monitored an updated via an application. The patient can input additional data (e.g., weight, age, height, odd symptoms, environmental conditions, etc.), and a health care provider can input additional data (e.g., new research, test results, etc.). This additional data can be used by the machine learning model to determine a seizure risk, seizure baseline, and seizure plan.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method, comprising:
receiving at a first processing resource, first signaling from a radio in communication with a second processing resource configured to monitor patient health data of a patient;
receiving at the first processing resource, second signaling from a radio in communication with a third processing resource configured to monitor health provider data associated with seizures;
receiving at the first processing resource, third signaling from a radio in communication with a fourth processing resource configured to monitor environmental data associated with the patient;
writing from the first processing resource to a memory resource coupled to the first processing resource data that is based at least in part on a combination of the first signaling, the second signaling, and the third signaling;
determining, at the first processing resource or a different, fifth processing resource, a seizure baseline for the patient and a seizure risk for the patient based on the first signaling, the second signaling, and the third signaling;
identifying, at the first processing resource or the different, fifth processing resource, output data representative of a seizure plan for the patient based at least in part on input data representative of written information, the seizure baseline, and the seizure risk stored in a portion of the memory resource or other storage accessible by the first processing resource; and
transmitting the output data representative of the seizure plan via fourth signaling sent via a radio in communication with a sixth processing resource of a computing device accessible by the patient.

2. The method of claim 1, wherein identifying the output data representative of the seizure plan comprises utilizing a trained machine learning model to identify the output data representative of the seizure plan based on data associated with the first signaling, the second signaling, the third signaling, the seizure baseline, the seizure risk, and previously received signaling and associated data associated with previous seizure plans.

3. The method of claim 1, wherein determining the seizure baseline and the seizure risk comprises utilizing a trained machine learning model to determine the seizure baseline and the seizure risk based on data associated with the first signaling, the second signaling, the third signaling, and previously received signaling and associated data associated with previous seizure plans.

4. The method of claim 1, wherein determining the seizure baseline comprises determining levels of each one of a plurality of factors associated with the patient at which a seizure is most likely to occur.

5. The method of claim 1, wherein determining the seizure risk comprises determining a likelihood at a particular period in time that the patient will have a seizure.

6. The method of claim 1, wherein identifying the output data representative of the seizure plan comprises:
identifying an alert to transmit to the computing device of the patient; and
identifying a proposed action and associated instructions to reduce a seizure risk of the patient, a proposed action and associated instructions to stay at or below a seizure baseline of the patient, or both.

7. The method of claim 1, further comprising updating the seizure baseline in response to receiving at the first processing resource additional first signaling, second signaling, third signaling, or any combination thereof and based at least in part on feedback received at the first processing resource associated with outcomes of the output data representative of the seizure plan.

8. The method of claim 1, further comprising:
receiving at the first processing resource via an application of the computing device accessible by the patient or a different mobile device of the patient, manual input from the patient comprising personal patient data, patient health data, environmental data, health care provider data, or a combination thereof; and
writing from the first processing resource to the memory resource coupled to the first processing resource data that is based at least in part on a combination of the first signaling, the second signaling, the third signaling, and the manual input.

9. A non-transitory machine-readable medium comprising a processing resource in communication with a memory resource having instructions executable to:
receive at a processing resource, the memory resource, or both, a plurality of input data from a plurality of sources, the plurality of sources comprising at least two of: a mobile device of a patient, a medical device, a health care provider database, a portion of the memory resource or other storage, manually received input, and environmental sensors;
write from the first processing resource to the memory resource the received plurality of input data;
identify at the first processing resource or a second processing resource, output data representative of a seizure plan including a proposed action to reduce a seizure risk of the patient, a proposed action to stay at or below a seizure baseline of the patient, or both, based at least in part on input data representative of the data written from the first processing resource;
transmit the output data representative of the seizure plan to the mobile device of the patient via signaling sent via a radio in communication with a third processing resource of the patient's mobile device.

10. The medium of claim 9, further comprising the instructions executable to identify the output data representative of the seizure plan based at least in part on generic seizure patient information and generic seizure treatment information stored in a portion of the memory resource or other storage accessible by the first processing resource.

11. The medium of claim 9, further comprising the instructions executable to identify the output data representative of the seizure plan based at least in part on patient medical history information stored in a portion of the memory resource or other storage accessible by the first processing resource.

12. The medium of claim 9, wherein the plurality of input data comprises patient health data, health care provider data, environmental data, or any combination thereof.

13. The medium of claim 9, further comprising the instructions executable to identify at the first processing resource or the second processing resource output data representative of the seizure plan using a trained machine learning model.

14. The medium of claim 9, wherein the instructions executable to transmit the output data representative of the seizure plan further comprise instructions executable to transmit an alert to the mobile device of the patient of the seizure risk, the proposed action to reduce the seizure risk of the patient, the proposed action to stay at or below the seizure baseline of the patient, or any combination thereof.

15. A non-transitory machine-readable medium comprising a first processing resource in communication with a memory resource having instructions executable to:
receive at the first processing resource, the memory resource, or both, patient health data via first signaling configured to monitor patient health data, via second signaling sent via a radio in communication with a processing resource of a mobile device of the patient, or both;
receive at the first processing resource, the memory resource, or both, health care provider data via third signaling configured to monitor health care provider data including generic seizure patient information and generic seizure treatment information;
receive at the first processing resource, the memory resource, or both, environmental data via fourth signaling configured to monitor environmental data including lighting, screen time, diet, humidity, temperature, or any combination thereof;
write from the first processing resource to the memory resource the patient health data, heath care provider data, and environmental data;
determine, at the first processing resource or a second processing resource, a seizure risk of the patient and a seizure baseline for the patient using a trained machine learning model, input data representative of the written patient health data, the written heath care provider data, and the written environmental data;
identify, at the first processing resource or a second processing resource, output data representative of a seizure plan for the patient using the trained machine learning model, input data representative of the written patient health data, the written health care provider data, and the written environmental data, and input data representative of the seizure risk and the seizure baseline; and
transmit, via a radio, the output data representative of the seizure plan to the patient, a health care provider, or any combination thereof.

16. The medium of claim 15, wherein the patient health data, the health care provider data, and the environmental data carry different weights within the trained machine learning model.

17. The medium of claim 15, further comprising a database of generic seizure information that is part of the memory resource or other storage communicatively coupled to the medium that comprises generic seizure symptoms and associated diagnoses and treatments.

18. The medium of claim 15, wherein the instructions executable to receive the patient health data via first signaling configured to monitor patient health data comprise instructions executable to receive the patient data via signaling from a health sensor, health monitor, wearable device, or mobile device of the patient.

19. The medium of claim 15, wherein the instructions executable to identify the output data representative of the seizure plan comprise instructions executable to:
determine an alert to transmit to the mobile device of the patient of the seizure risk;
determine an alert to transmit to a computing device of a health care provider of the seizure risk;
determine an alert to transmit to a mobile device of an authorized user of the seizure risk;
determine a proposed action to reduce the seizure risk of the patient;
determine a proposed action to stay at or below the seizure baseline of the patient; or
any combination thereof.

20. The medium of claim 15, wherein the patient health data comprises health symptoms, a health event, personal health information of the patient, identifying information of the patient, a location of the patient, data collected by a health monitor, manually input data of the patient, or any combination thereof.
